(19) United States
(12) Patent Application Publication
(10) Pub. No.: US 2026/0060972 A1
(43) Pub. Date: Mar. 5, 2026

(54) Quantitative Method of Determining a Mental Status Based on DRD1 and/or DRD5, and its Application Thereof

(71) Applicant: Xiandong Shi, Port Washington, NY (US)

(72) Inventors: Xiandong Shi, Port Washington, NY (US); Xingguo Cheng, Flushing, NY (US); Yue Ji, Flushing, NY (US); Zhanqing Yan, Glen Head, NY (US); Jianyou Tan, Short Hills, NY (US)

(21) Appl. No.: 19/100,897

(22) PCT Filed: Aug. 3, 2023
(86) PCT No.: PCT/US2023/071631

§ 371 Date: Feb. 3, 2025

Related U.S. Application Data
(60) Provisional application No. 63/370,375, filed on Aug. 3, 2022.

(30) Foreign Application Priority Data
Aug. 3, 2022 (US) 63370375

(51) Int. Cl. A61K 31/4515; C12Q 1/6883

(52) U.S. Cl. CPC A61K 31/4515; C12Q 1/6883; C12Q 2600/156; C12Q 2600/158

(57) ABSTRACT

A method of treatment of mental disorder of a subject includes the steps of: normalizing an expression of both DRD1 and DRD5, which belong to DRD1-like family, of the subject to a preset standard level. The DRD1/5 level and expression can be increased or decreased bidirectionally by targeting an upstream promoter sequence of DRD1/5. The promoter activity of DRD1/5 can be attenuated by using an antipsychotic which directly targets the DRD1/5 promoter. The antipsychotics being used are typical and/or atypical antipsychotic medications such as Haloperidol,

Risperidone, Clozapine and Cariprazine. The treatment is also useful for the subject with an impaired constitutive expression of DRD1/5 gene. The method can be used to treat schizophrenia, autism, depression, mania and bipolar disorders by normalizing the DRD1/5 expression level. The promoter gene sequencing of DRD1 and/or DRD5 can also be used for quantitative mental health screening through comparing with a standard sequence database.

### BACKGROUND OF THE PRESENT INVENTION

### Field of Invention

The present invention relates to Dopamine receptor D1, also known as DRD1, and Dopamine receptor D5, also known as D1BR or DRD5, and more particularly to a quantitative method of determining a mental status of a human subject such as psychotic patients based on DRD1 and/or DRD5, and its application thereof.

### Description of Related Arts

Currently, there is no quantitative method of determining a mental status of a human subject. For example, psychosis, which interferes with the ability to function, is determined by symptoms such as delusions and hallucinations. The etiology of psychotic disorders is not known and the commercially available antipsychotic medications have no common modes of action.

The diagnosis of mental disorders relies heavily on signs and symptoms of the human subject. Examples include feeling sad or down, confused thinking or reduced ability to concentrate, excessive fears or worries, or extreme feelings of guilt, mood changes of highs and lows, withdrawal from friends and activities, significant tiredness, problems sleeping, delusions, paranoia or hallucinations, excessive anger, hostility or violence, and etc. Sometimes, symptoms may include stomach pain, back pain, headaches, or other unexplained aches and pains. It is very difficult to determine the mental status of a human subject because there are a great number of possible symptoms which are mostly based on the behavior and the thinking of the human subject.

At present, there is some linkages between schizophrenia and genetic. However, no single gene is thought to be responsible and it is only determined that people with family members who have psychotic disorder are at an increased risk. The cause or linkage remain unknown.

Existing antipsychotical medications are typically DRD2 antagonist. However, the current understanding of whether DRD2 is involved in actions of antipsychotical medications remains controversial. Serotonin and dopamine are two common neurotransmitters, which are believed to have direct impact on mental disorders.

### SUMMARY OF THE PRESENT INVENTION

An object of the present invention is to provide a method of quantitative determination of mental status of a human subject based on DRD1 and/or DRD5, and its application and use thereof.

Another object of the present invention is to provide antipsychotic medications to normalize chemically altered DRD1 expression.

Another object of the present invention is to provide antipsychotic medications similarly impact DRD1 and DRD5 expression, which is capable of normalize or stabilize both up-regulated and down-regulated DRD1 and DRD5 expression to normal expression level.

Another object of the present invention is to provide antipsychotic medications directly target DRD1 and/or DRD5 promoter sequences.

Another object of the present invention is to provide antipsychotic medications which involves a new mode of actions when compared to conventional antipsychotic medications, therefore it is possible to develop new medications based on maintaining a normalized expression level of DRD1 and DRD5.

Another object of the present invention is to provide antipsychotic medications to normalizing DRD1 expression by targeting 5′-regulatory elements.

Another object of the present invention is to provide a method of treatment for depression and manic.

Another object of the present invention is to provide a method of treatment for restoring both positive and negative symptoms in psychotic disorders by neutralizing a critical process which involves bidirectional regulation of DRD1 and DRD5 expression.

Additional advantages and features of the invention will become apparent from the description which follows, and may be realized by means of the instrumentalities and combinations particular point out in the appended claims.

According to the present invention, the foregoing and other objects and advantages are attained by a method of treatment of mental disorder of a subject, which comprises the steps of:
normalizing a DRD1 level and a DRD1 expression of the subject to a preset standard level.

Preferably, the DRD1 level and a DRD1 expression can be increase or decrease bidirectionally by targeting an upstream promoter sequence of DRD1. The promoter activity of DRD1 can be attenuated by using an antipsychotic which directly targets the DRD1 promoter. The antipsychotic being used can be one or more of typical and atypical antipsychotic medications such as Haloperidol, Risperidone, Clozapine and Cariprazine. The treatment is also useful for the subject with an impaired constitutive genetic expression of DRD1. The method can be used to treat schizophrenia, autism, depression, mania and bipolar disorders by normalizing the DRD1 expression level.

According to another aspect of the present invention, a method of treatment of mental disorder of a subject comprises the steps of:
bidirectionally normalizing an overall expression level of DRD1 and DRD5 of the subject to a preset standard level.

Preferably, the method further comprises the steps of: attenuating a promoter activity of DRD1/DRD5 by using an antipsychotic which directly targets the DRD1 and DRD5 promoter.

Preferably, the method further comprises the steps of: providing a sequence database in which polymorphism of DRD1 and DRD5 promoter sequences are related to medication efficacy; and personizing medication screening of antipsychotic drugs regarding efficacy based on the sequence database.

According to another aspect of the present invention, a screening method of mental disorder of a subject comprises the steps of: carrying out promoter gene sequencing of DRD1 and DRD5 of the subject to obtain a sample sequence; and comparing the sample sequence to a standard sequence database of human DRD1/DR5 promoter to determine if the object is suffered from a mental disorder.

Preferably, the polymorphism of DRD1/DRD5 promoter sequences which are related to clinically diagnosed mental disorders are included in the standard sequence database.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1A illustrates a relative DRD1 Gene mRNA Expression under Haloperidol (10 μM), SCH-23390 (10 μM), and SKF-81297 (10 μM) Treatment on SH-SY5Y Cell.

FIG. 1B illustrates the results of protein expression of DRD1 under Haloperidol Treatment on SH-SY5Y Cell.

FIG. 1C illustrates a relative protein expression of DRD1 under Haloperidol Treatment on SH-SY5Y Cell.

FIG. 2A illustrates a relative DRD1 mRNA Expression of SCH-23390 Treatment on SH-SY5Y Cell.

FIG. 2B illustrates the results of protein expression of DRD1 under SCH-23390 Treatment on SH-SY5Y Cell.

FIG. 2C illustrates a relative protein expression of DRD1 on SH-SY5Y Cell

FIG. 2D ICC staining SCH treated SHSY5Y (36 hrs) under different treatment.

FIG. 2E illustrates the percentage of positive H-DRD1 cells on SHSY-5Y cells under different treatment.

FIG. 3A illustrates relative DRD1 mRNA Expression of SKF-81297 Treatment on SH-SY5Y Cell.

FIG. 3B illustrates the results of protein expression of DRD1 under SKF-81297 Treatment on SH-SY5Y Cell.

FIG. 3C illustrates a relative protein expression of DRD1 on SKF-81297 Treatment on SH-SY5Y Cell.

FIG. 3D illustrates an ICC staining SKF treated SHSY5Y under different treatment.

FIG. 3E illustrates the percentage of positive H-DRD1 cells on SHSY-5Y cells under different treatment

FIG. 4A illustrates a relative DRD1 promoter activity under SCH-23390 Treatment on SH-SY5Y Cell

FIG. 4B illustrates a relative DRD1 promoter activity under SKF-81297 Treatment on SH-SY5Y Cell.

FIG. 5A and 5B illustrates studies the regulation of DRD1 promoter activity by Risperidone. FIG. 5A illustrates a relative Luciferase activity of DRD1 under treatment of SCH and Risperidone. (Risperidone has partial DRD1 antagonist effect with co-treatment of SCH) FIG. 5B illustrates a relative Luciferase activity of DRD1 under treatment of SKF and Risperidone. (Risperidone has partial DRD1 antagonist effect with co-treatment of SKF)

FIG. 6A and 6B illustrates studies the regulation of DRD1 promoter activity by Clozapine. FIG. 6A illustrates a relative Luciferase activity of DRD1 under treatment of SCH and Clozapine (Clozapine has partial DRD1 antagonist effect with co-treatment of SCH) FIG. 6B illustrates a relative Luciferase activity of DRD1 under treatment of SKF and Clozapine (Clozapine has partial DRD1 antagonist effect with co-treatment of SKF)

FIG. 7A and 7B illustrates studies the regulation of DRD5 mRNA expression. FIG. 7A illustrates relative DRD5 mRNA expression with treatment of SCH-23390 on SH-SY5Y cell. FIG. 7B illustrates relative DRD5 mRNA expression with treatment of of SKF-81297 and H on SH-SY5Y cell.

FIG. 8 illustrates the relative mRNA expression of DRD1 and DRD5 in SHSY5Y cells.

### DETAILED DESCRIPTION OF THE PREFERRED EMBODIMENT

According to the present invention, DRD1 and DRD5, especially its protein expression levels, play critical roles in the maintenance of active status of each individual functional cells, such as neuronal, muscular, and endothelial cells.

Dopamine receptor D1 (DRD1), encoded by DRD1 gene, is one of the two members of DRD1-like receptor family which includes DRD1 and DRD5. DRD1 is mostly abundant in the central nervous system. DRD1 plays important roles in the memory, learning, addiction, reward system, behavior appearance, and the development of neurons. DRD1 is one of the most abundant dopamine receptors in the human brain and plays important roles in the etiology of psychiatric disorders, including schizophrenia, autism, depression, mania and bipolar disorders.

DRD1 and DRD5 belong to D₁-like family of dopamine receptors. Functionally, DRD1 and DRD5 are compensatory. Accordingly, DRD1 and DRD5 can compensatorily and coordinately maintain mental status. Preferably, both DRD1 and DRD5 are involved in the maintenance of mental status, which can help to sustain genetic and functional stability and can further explain the diversity of mental disorders.

According to the present invention, DRD1 dysfunction is associated with psychological disorders. Psychotic diseases, including bipolar disorder, depression, mania, and schizophasia, are essentially promoter disorders. Psychosis is primarily due to promoter-associated gene expression disorders, especially DRDs. Accordingly, antipsychotics may function by normalizing chemically altered DRD1 expression back to constitutive levels. The action of antipsychotics is two-ways, which can normalize both up-regulated and down-regulated DRD1 expression. Antipsychotics play a “hormesis” effect by normalizing DRD1 expression.

According to the present invention, there are two possible mechanisms for the regulation of DRD1 expression as follows:
(a) There are two individual elements in DRD1 promoter sequence: one is responsible for attenuation of up-regulated DRD1 expression; the other one contributes to antipsychotic's reversal effect against down-regulated DRD1 expression.
(b) There exists a specific element in the DRD1 promoter. When they target this sequence, antipsychotics will block chemically altered DRD1 expression machinery and thus stabilize the DRD1 expression to constitutive expression level.

It is worth mentioning that clinically, existing antipsychotic medications are more effective in bipolar disorders than Schizophrenia. According to the principles of the present invention, constitutive genetic expression of DRD1 is significantly impaired, even after antipsychotic medications treatment. The expression of DRD1 following antipsychotic medication treatment in Schizophrenia patients cannot be returned to normal DRD1 expression range. As a result, the existing antipsychotic medications are not effective in schizophrenia's treatment.

DRD1 and DRD5 together coordinate mental status. The overall expression levels of DRD1 and DRD5 are managed at normal range to sustain normal mental status.

In psychotic patients, the overall expression levels of DRD1 and DRD5 are out of normal ranges. Specifically, when the overall expression levels of DRD1 and DRD5 are very high, the patients show depression and negative symptoms. In contrast, when the overall expression levels of DRD1 and DRD5 are very low, the patients depict manic and positive symptoms. In addition, when the promoters of DRD1 and DRD5 are prone to internal or external factors, which leads to readily drift of their expression, the patients depict bipolar disorders.

Promoter sequencing of human DRD1 and DRD5 can be used to diagnose and screen mental disorders.

Our conventional understanding of modes of action of antipsychotic medications is primarily based on DRD2. For instance, all antipsychotics reduce dopaminergic neurotransmission. The primary modes of action of most first-and second-generation antipsychotics appears to be postsynaptic blockade of DRD2-like receptors in the brain. In addition, most second-generation antipsychotics also inhibit serotonin receptors 5HT, such as 5HT1A/2A.

As the present invention provides that the overall expression levels of DRD1 and DRD5 can serve as an objective factor of mental status, it is now possible to correlate the expression levels of DRD1 and/or DRD5 with mental healthiness directly. Thus, the effect of antipsychotic medications on DRD1 and DRD5 can be further studied for drug development.

For example, DRD1 expression is dramatically impaired in patients with schizophrenia. The importance of certain DRD1 promoter gene sequences can then be used to modulating human mental disorders through regulating the level of DRD1 and DRD5.

According to a first preferred embodiment of the present invention, a method of treatment of mental disorders comprises the steps of: normalizing human DRD1 level and expression to restore to normal level; and/or normalizing human DRD5 level and expression to restore to normal level.

Preferably, the method can further include the steps of: increasing or decreasing the DRD1 constitutive expression of the subject by targeting an upstream promoter sequence of DRD1; and/or increasing or decreasing the DRD5 constitutive expression of the subject by targeting an upstream promoter sequence of DRD5.

Preferably, the method can further include the steps of: attenuating a promoter activity of DRD1 or DRD5 by using an antipsychotic which directly targets the DRD1 or DRD5 promoter.

Human DRD1 and DRD5 genes essentially contribute to the maintenance of mental status. Genetic and/or epigenetic alterations in human DRD1 and DRD5promoter sequences result in variations in their expression. Antipsychotic drugs directly target the human DRD1 and DRD5 promoter to normalize exogenously or endogenously altered human DRD1 and DRD5 expression, leading to their therapeutic effects.

Preferably, the method of treatment of mental disorder includes both medications approaches or non-medication approaches to normalizing human DRD1 expression to restore to within a preset normal level.

Preferably, antipsychotics is used to normalize the expression level of DRD1 and/DRD5. The antipsychotics can be selected from different categories of antipsychotic medications. Typical antipsychotic Haloperidol and atypical antipsychotics Risperidone, Clozapine and Cariprazine are selected. Therefore, these medications can mechanistically represent most antipsychotics.

According to a second preferred embodiment of the present invention, a method of quantitative screening and testing for psychiatric disorders comprises the steps of: carrying out promoter sequencing of DRD1 and/or DRD5 genes by applying extracted genomic DNA samples of a human subject; then screening and comparing the sequence to a human DRD1 and/or DRD5 promoter sequence database to determine if the human subject is suffered from a psychiatric disorder.

If at least one variation is found in DRD1 or DRD5, the human subject is confirmed to have a psychiatric disorder. If at least two variation is found in DRD1 or DRD5, the human subject is confirmed to have a psychiatric disorder of increased level (more serious mental disorder).

The human DRD1/DRD5 promoter sequence database is established in a sequencing project. The polymorphism of DRD1/DRD5 promoter sequence is related to clinically diagnosed mental disorders in the sequence database. In addition, this sequence database will be applied in prenatal and parental screening to predict the odd of mental disorders in newborns. The variations in DRD1/DRD5 promoter sequences are associated with mental disorders.

In addition, the method further includes the steps of: personizing medication screening of antipsychotic drugs regarding efficacy based on the database in which polymorphism of DRD1 and DRD5 promoter sequences is related to medication efficacy.

According to the present invention, the overall expression levels of DRD1 and DRD5 are maintained at a preset normal range for treatment of mental disorders. Therefore, by screening known chemical libraries, potential drug candidates that can normalize altered DRD1 and DRD5 expression can be selected for new drug development.

According to another preferred embodiment of the present invention, a quantitative screening method of mental disorder of a subject, comprises the following three steps:
Step 1: To carry out genomic sequencing of DRD1 and DRD5 genes (including 7-kb 5′-upstream sequence, protein coding sequence, and 2-kb 3′-downstream sequence) of the human subject to establish the relationship between the mental disorders and the variant distribution database (including the type, location and frequency of each gene variant) in the DRD1 and DRD5 genes.
Step 2: To sort out the variants that are highly related to each mental disorder to develop the target gene sequence of human DRD1 and DRD5 genes, which can predict the risk of developing mental disorders.
Step 3. To perform the target gene sequence of DRD1 and DRD5 genes in new subject to determine the risk of developing a mental disorder in the subject.

In other words, a quantitative method of screening test of human DRD1 and DRD5 expression in the cells (such as mucosal cells, skin cells, blood cells, etc.) of human subjects can also be provided to establish the normal ranges of human DRD1 and DRD5 expression in the cells. If a new subject's DRD1/5 expression is out of normal range in that cell, it serves as an indicator of mental status of the subject.

Haloperidol, a typical antipsychotic, is commonly prescribed to treat schizophrenia. Therapeutic effects of Haloperidol are thought to be due to its inhibition on DRD2 activity in conventional treatments. By studying the relationship between Haloperidol treatment and DRD1 and DRD5, instead of that of DRD2, can establish a direct and effective correlation for effective quantitative treatment.

### Experiment 1: Haloperidol-DRD1 Project

In general, the action of a drug receptor relies upon its binding affinity to its ligands (Km) and its total expression (Vmax). The binding affinity is determined by its protein configuration. In contrast, the expression of receptor is genetically and epigenetically affected.

In order to illustrate the roles of DRD1 in the modes of act of antipsychotic medications, in vitro studies to determine the regulation of DRD1 expression by several typical and atypical antipsychotic medications including Haloperidol, Clozapine, Risperidone and Cariprazine in the presence of DRD1 agonists or antagonists are carried out.

This study is designed to determine whether Haloperidol, Risperidone, or Clozapine, which are the widely prescribed antipsychotics, can normalize chemically altered human DRD1 expression in cultured human SH-SY5Y neuroblastoma cells.

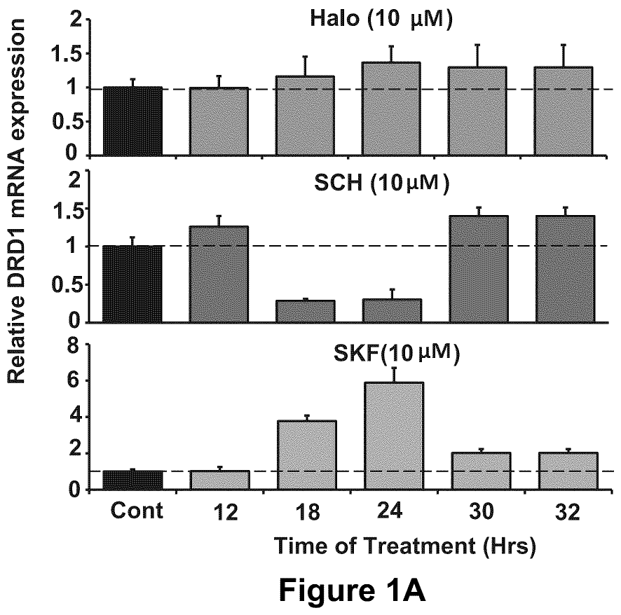

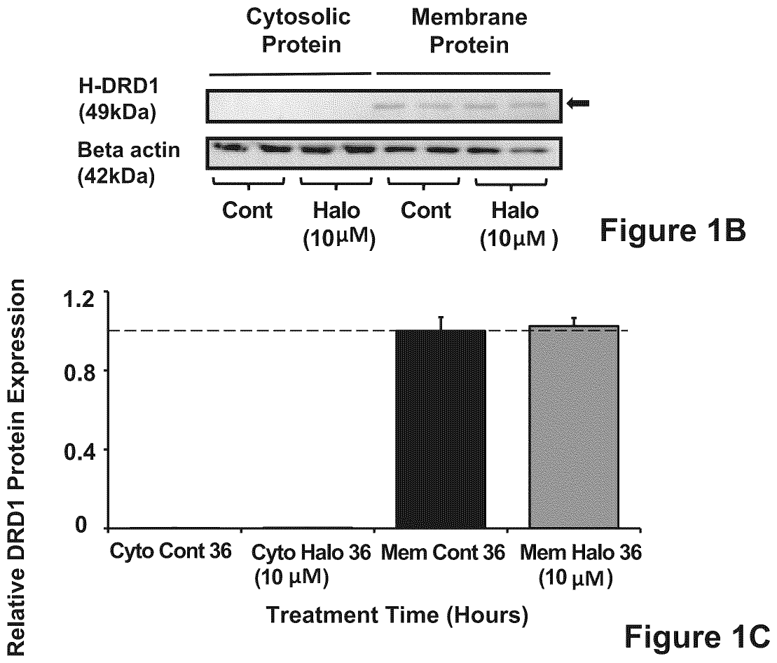

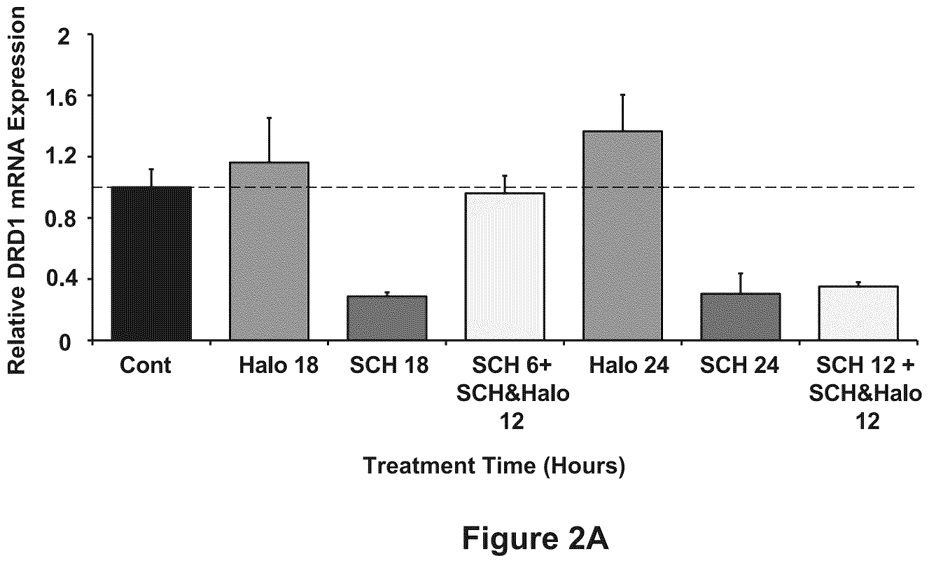

Referring to FIG. 1A, the relative DRD1 Gene mRNA Expression under treatment of Haloperidol (10 μM), SCH-23390 (10 μM), and SKF-81297 (10 μM) Treatment in SH-SY5Y Cell is illustrated.

SCH-23390, also known as halobenzazepine, is a synthetic compound that acts as a D1 receptor antagonist. SKF-81297, a synthetic compound of the benzazepine chemical class, acts as a selective dopamine D1/D5 receptor full agonist. SH-SY5Y cell is a cloned subline of a human neuroblastoma cell line.

According to this experiment, Haloperidol can normalize chemically altered human DRD1 expression in cultured human SH-SY5Y neuroblastoma cells.

### Materials and Methods

Haloperidol was purchased from Sigma-Aldrich (cat. #H1512-10G, Saint Louis, MO). SCH23390 hydrochloride (cat. #09-251-0) and SKF81297 hydrobromide (cat. #14-471-0) were purchased from FISHER SCIENTIFIC (Hanover Park, IL). Lipofectamine reagent was purchased from FISHER SCIENTIFIC (cat. #NC0904455, HANOVER PARK, IL). Both mem-PER Plus membrane protein extraction kit (cat. #89842) and NE-PER Nuclear and cytoplasmic extraction reagent kit (cat. #78833) were purchased from Fisher Scientific (Hanover Park, IL). Zymo Plasmid Miniprep kit (cat. #D4036) and DNA Clean & Concentrator kit (cat. #D4013) were purchased from Zymo Research Corporation (Irvine, CA). All other chemicals were purchase from Thermo Fisher Scientific Inc. (Waltham, MA) unless otherwise stated.

The anti-Dopamine Receptor D1 (DRD1) antibody was obtained from Abcam (cat. #ab216644, Cambridge, MA). Both Pierce Goat Anti-Rabbit IgG (H+L) Biotin Conjugated secondary antibody (1:5,000) and Pierce High Sensitivity Streptavidin-HRP 1:5,000 (cat. #21130) were purchased from Thermo Fisher Scientific (Waltham, MA).

Cell culture study: Human SH-SY5Y neuroblastoma cell line, originally purchased from ATCC (cat. #CRL-2266, Manassas, VA), was cultured in Eagle's Minimum Essential Medium (EMEM) (cat. #ATCC® 30-2003, ATCC, Manassas, VA) supplemented with 10% heat-inactivated fetal bovine serum (Atlanta Biologicals; Norcross, GA). The CO₂ cell incubator was maintained at 37° C. with 5% CO₂ in a humidified atmosphere. The cells treated in 6-well plates were harvested for mRNA analysis by performing quantitative real time-PCR assay. Cells were seeded into 6-well plates at a density of 3×10⁵/well when the drug treatment is more than 24 hrs, and 4×10⁵/well when the drug treatment is less than 24 hrs. The cells treated in T-75 Flasks were harvested for protein analysis by performing western blotting. Cells were seeded into T-75 Flasks at a density of 3×10⁶/Flask (65% confluency). Cells, which were seeded into 96-well plates at a density of 2.5×10⁴/well, were used in the promoter analysis by performing Dual-Luciferase reporter assay.

Time-response study. We selected 10 μM Haloperidol, 10 μM SCH23390, or 10 μM SKF81297 as starting doses in the time-response studies. The cultured SH-SY5Y cells were treated with complete EMEM medium containing 10 μM Haloperidol, 10 μM SCH23390, or 10 μM SKF81297 for 12, 18, 24, 30, or 32 hrs, respectively. The controls received DMSO only. After each time period, the cells were harvested and processed for mRNA analysis of human DRD1 gene.

Haloperidol and SCH23390 co-treatment study. The cultured SH-SY5Y cells were pretreated with complete EMEM medium containing 10 μM SCH23390 for 6 hrs or 12 hrs. Then, the cells were treated with complete EMEM medium containing 10 μM Haloperidol and 10 μM SCH23390 for 12 hrs or 24 hrs. After treatment, the cells were harvested and processed for mRNA and protein analysis of human DRD1 gene.

Haloperidol and SKF81297 co-treatment study. The cultured SH-SY5Y cells were pretreated with complete EMEM medium containing 10 μM SKF81297 for 6 hrs or 12 hrs. Then, the cells were treated with complete EMEM medium containing 10 μM Haloperidol and 10 μM SKF 81297 for 12 hrs or 24 hrs. After treatment, the cells were harvested and processed for mRNA and protein analysis of human DRD1 gene

### Total RNA Extraction and Complementary DNA (cDNA) Preparation

RNA extraction was performed by using TRIzol™ RNA extraction reagent (cat. #15596026; Life Technologies; Carlsbad, CA). The concentration and purity of total RNAs was determined by Biospectrometer (Eppendorf, Hauppauge, NY) at 260 nm. Only RNA samples with an A260/A280 ratio between 1.8-2.0 were used for cDNA synthesis. Briefly, 3 μg total RNAs were reversely transcribed into cDNA using Random and Oligo 9dT) Primers (cat #PR-C1181, Fisher Scientific, Hanover Park, IL), Oilgo dT (cat #FERSO131, Fisher Scientific, Hanover Park, IL), and SuperScript II reverse transcriptase (Life Technologies, Carlsbad, CA) following the manufacturer's instructions. The synthesized cDNA samples were stored at −20° C.

### Quantitative Real Time-PCR (qRT-PCR) Assay

Each cDNA sample, in duplicate, was placed into 96-well plates and mixed with SYBR Select Master Mix (Life Technologies, Carlsbad, CA) and gene-specific primer set. In general, each SYBR reactions in each well contains 5 ml SYBR Select Master Mix, 0.2 μl 10 μM gene-specific primer set, 1.8 μl deionized distilled water, and 3 μl cDNA samples. The qRT-PCR assay was performed in an AriaMx qRT-PCR system (Agilent Technologies; Santa Clara, CA). The relative mRNA expression of each individual gene was calculated according to the comparative delta-delta CT method. The results were expressed as a relative fold, normalized to the expression of 18s rDNA. All quantitative RT-PCR primers were designed using PubMed Primer-BLAST or Primer3 software (NIH) and synthesized by Eurofins (Eurofins MWG Operon USA, Louisville, KY).

The sequences of RT-PCR primers of human DRD1 are as follows:
Gene human DRD1, forward primer: [Sequence ID: 1] 5′-CCATCACACAAAACGGTCAG-3′, reverse primer: [Sequence ID: 2] 5′-GTGTGTTGGAAAGCAGCAGA-3′
Gene 18s rDNA, NM number NR_003278.3, forward primer [Sequence ID: 3] 5′-GCAATTATTCCCCATGAACG-3′; reverse primer [Sequence ID: 4] 5′-GCCTCACTAAACCATCCAA-3′

### Western Blotting

Human SH-SY5Y neuroblastoma cells were grown in T-75 flasks to 65% confluency and then treated with chemicals as described above. The membrane proteins were extracted using the mem-PER Plus membrane protein extraction kit (ThermoFisher Scinetific, Waltham, MA). The protein concentrations were semi-quantified spectrophotometrically at 280 nm. The protein samples were prepared at a density of 30 mg/well mixed with loading buffer, heated at 95° C. for 5 minutes, and separate on a 12% SDS-polyacrylamide gel. Then, proteins were electro-transferred to a polyvinyl difluoride (PVDF) membrane. The membrane was then blocked in Tris-buffered saline (TBS) supplemented with 2.5% non-fat milk. After the block, the membrane was incubated with primary antibody (human DRD1 antibody, 1:1000) overnight at 4° C. Next day, the membrane was washed for 3 times using fresh TBS, then incubated with secondary antibody [goat anti-rabbit biotin-conjugated antibody (Abcam; Cambridge, MA) (1:5000) in TBS supplemented with 2.5% BSA for 2.5 hrs at room temperature. The membrane was briefly washed 3 times using fresh TBS, then incubated with Pierce High Sensitivity Streptavidin HRP-linked secondary antibody (1:5000) in TBS supplemented with 0.5% BSA for 30 minutes at room temperature. The membrane was rinsed 3 times using fresh TBS. The immunoreactive protein bands in the membrane were visualized via UVP Biospectrum Imaging system (Upland, CA) using with Immobilon Chemiluminescence reagents (Millipore, Billerica, MA). Protein bands analysis was performed using the ImageJ (NIH, Bethesda, MD). In addition, protein expression of Beta Actin, the loading control, was determined by using primary antibody (Beta Actin monoclonal antibody, 1:2000) and corresponding secondary antibody [Goat anti-Mouse IgG (H+L) Secondary Antibody, HRP 1:5000 (cat #31430)].

Immunocytochemistry (ICC) staining. Human SH-SY5Y neuroblastoma cells were seeded in chamber slides (1×10⁵/well) and then treated with chemicals as described above. Fixation and blocking were performed as we previously described (Bu et al., 2016). Briefly, cells were incubated with the antibody (1:100) against human DRD1 protein (1;1000) overnight at 4° C. Next day, chamber slides were incubated with 1:300 Pierce Goat Anti-Rabbit IgG (H+L) Biotin Conjugated secondary antibody (cat. #31820; Thermo Fisher Scientific Inc., where) for 4 hours at room temperature. Cells were then washed by 1×PBS and incubated with 1:400 Pierce High Sensitivity Streptavidin-HRP (cat. #21130; Thermo Fisher Scientific Inc., where) for one hour at room temperature. After final wash, color development was achieved using ImmPACT NovaRED Peroxidase (HRP) substrate (cat. #SK4805; Vector Laboratories, Inc.; Burlingame, CA) according to the manufacturer's instructions.

Cloning of human DRD1 Gene Promoters. Human DRD1 gene promoter sequences (172 bp, 312 bp, 473 bp, 961 bp, and 2772 bp upstream of transcriptional start site) were synthesized and engineered into pGL3-basic vector (cat. #E1751; Promega; Fitchburg, WI) at the polycloning sites between NheI and XhoI by GenScript (Piscataway, NJ). The sequences of recombinant pGL3 constructs were validated by GenScript (Piscataway, NJ) or Eurofins (Eurofins MWG Operon USA, Louisville, KY).

DNA Transfection and Dual-Luciferase Reporter Assay. The pGL3-basic vector (cat. #E1751; Promega; Fitchburg, WI) and the recombined pGL3 vectors containing various lengths of human DRD1 gene promoter were transfected into Human SH-SY5Y cells using Lipofectamine 2000 transfection reagent (cat. #11668027; Life Technologies, Inc.; Carlsbad, CA) following the manufacturer's instructions. In brief, cells were seeded into 96-well plates. Then, each well was transfected with 180-200 ng pGL3 plasmid DNAs (either empty pGL3-basic vector or recombinant pGL3 vectors containing human DRD1 gene promoter) and 10 ng of pRL-CMV Renilla luciferase control reporter vector (cat. #E2261; Promega; Madison, Wisconsin), and mixed with Lipofectamine 2000 (1:1, v/v). Vortex gently to mix the plasmids evenly in each well. Then, leave the plates in the incubator at a preset temperature for 24 hours. The transfection was stopped by replacing with freshly prepared DMEM medium. After the treatment, the medium was removed, and the well was rinsed with PBS once. Then the cells were lyzed. Dual-luciferase reporter assays were performed following the manufacturer's instructions (cat. #E1910; Promega; Fitchburg, WI). All the luminescence data for both firefly and Renilla luciferases were read by the luminometer (Promega). The relative firefly/Renilla luciferase activity value for each sample was calculated as in fold by comparing the mean value of the experiment groups to the control groups.

Statistical analysis All data are represented as mean±standard error (n=5-6/treatment). The comparison of the data from two treatment groups were analyzed by Student's t-test. The comparison of the data from more than two treatment groups were analyzed by one-way analysis of variance (ANOVA), followed by Duncan's post-hoc tests (Sigmaplot; Systat Software, Inc.; San Jose, CA). the data with p<0.05 was considered statistically significant.

### Results

Time response of DRD1 mRNA and protein expression by Haloperidol, SCH23390 and SKF81297 in SH-SY5Y cells. As shown in FIG. 1A, 10 μM of Haloperidol did not alter DRD1 mRNA expression at any time points after treatment. In contrast, 10 μM SCH23390 decreased DRD1 mRNA expression 18 and 24 hrs later, but not at other time points. 10 μM SKF81297 time-dependently increased DRD1 mRNA expression, reaching 6-fold 24 hrs later. Then, the expression of DRD1 mRNA expression decreased to basal level 30 hrs later and thereafter. DRD1 protein is not detectable in the cytosol. As shown in FIG. 1B, Haloperidol treatment did not alter DRD1 membrane protein level.

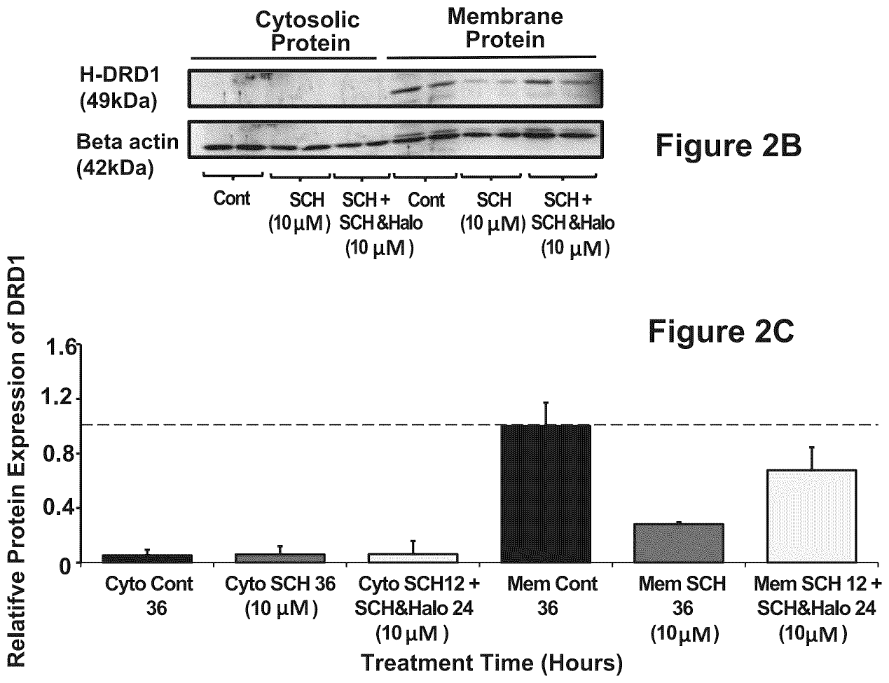

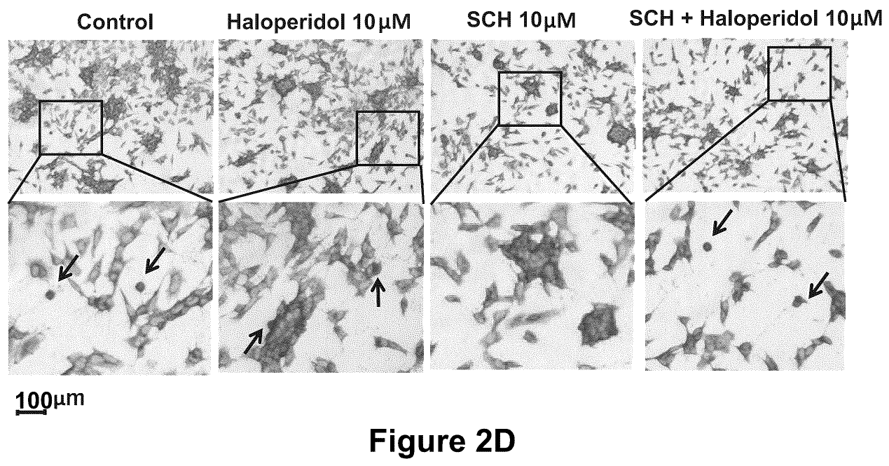

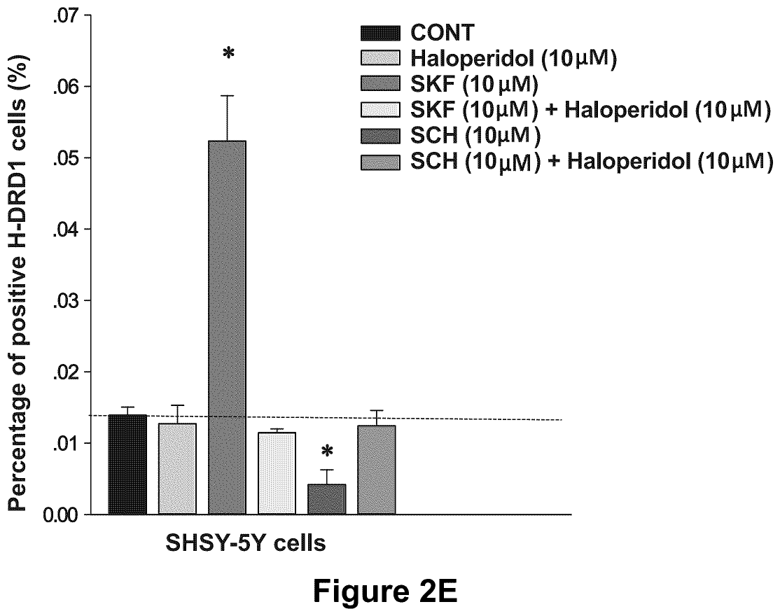

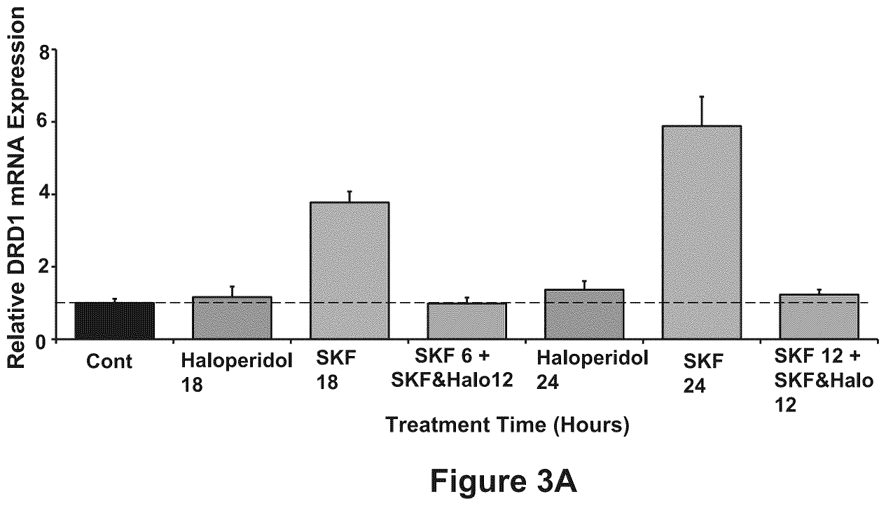

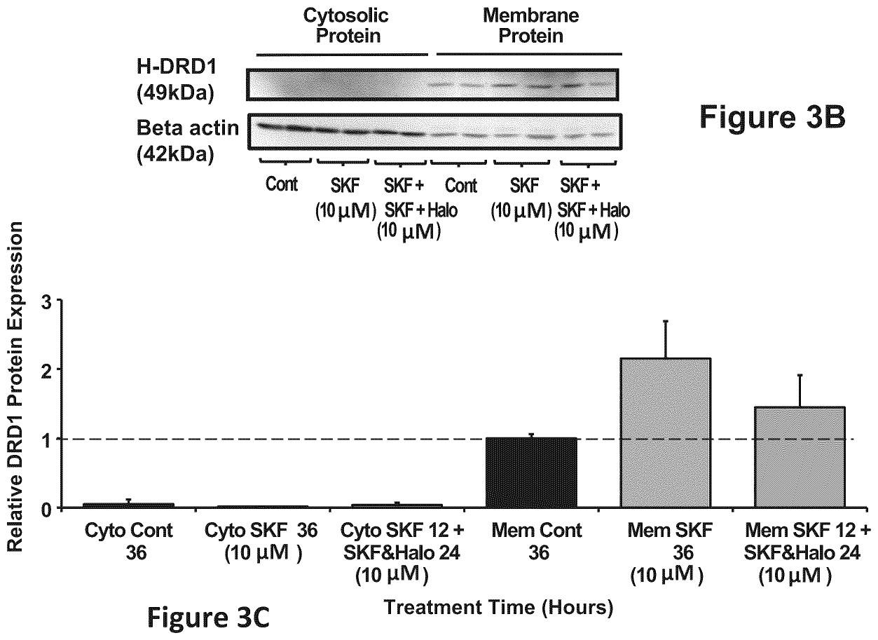

Referring to FIGS. 2A-2E of the drawings, the results show that Haloperidol reverses SCH23390-decreased DRD1 mRNA and protein expression in SH-SY5Y cells.

As shown in FIG. 2A, Haloperidol did not alter DRD1 mRNA expression. SCH23390 treatment for 18 hrs decreased DRD1 mRNA expression, which is completely reversed by 12 hrs Haloperidol co-treatment. In contrast, SCH23390 treatment for 24 hrs still markedly decreased DRD1 mRNA expression, which is not reversed by 12 hrs Haloperidol cotreatment. This is due to insufficient co-treatment time.

SCH23390 treatment for 36 hrs decreased DRD1 membrane protein more than 70%. Haloperidol co-treatment for 24 hrs markedly recover SCH-decreased DRD1 membrane protein to nearly 80% (FIG. 2C). DRD1-positive cells are shown as dark brown. As shown in FIG. 2D, Haloperidol did not apparently alter the counting of DRD1-positive cells. Whereas SCH significantly decreased the counting, which is reversed by Haloperidol cotreatment (FIG. 2D and FIG. 2E).

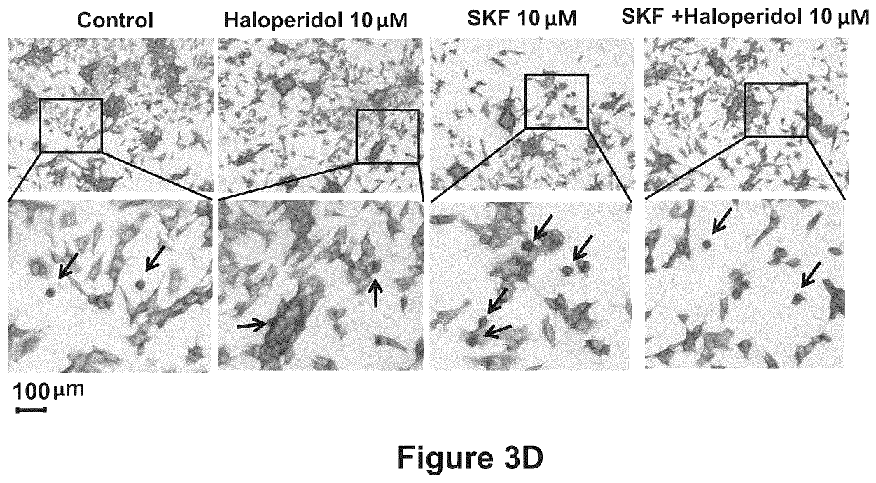

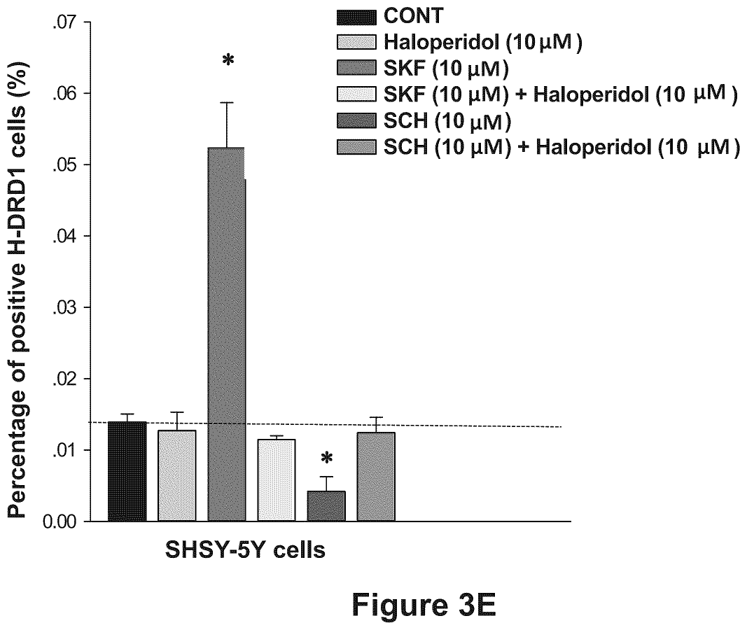

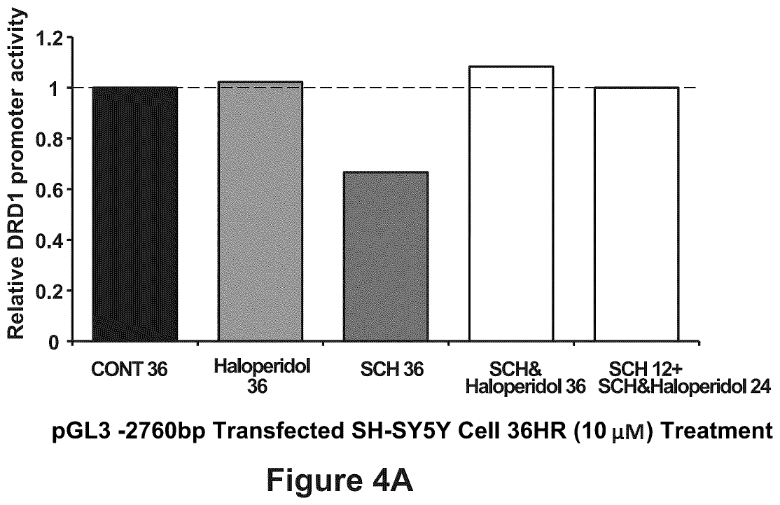

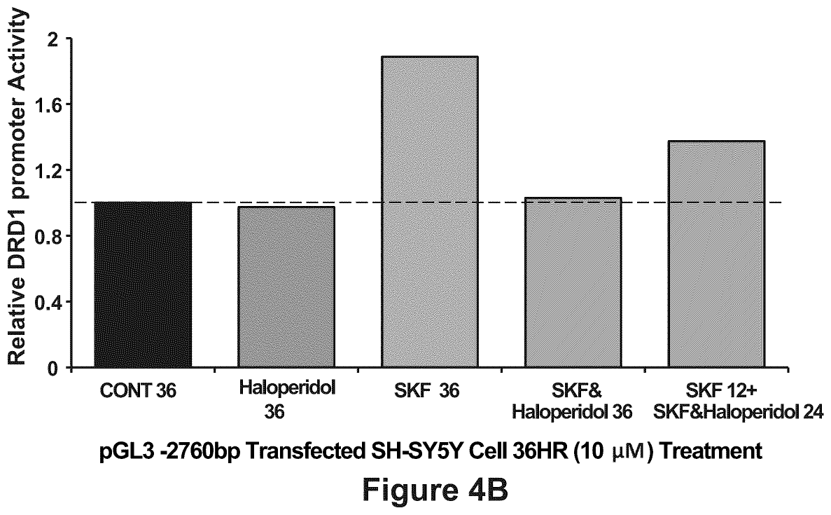

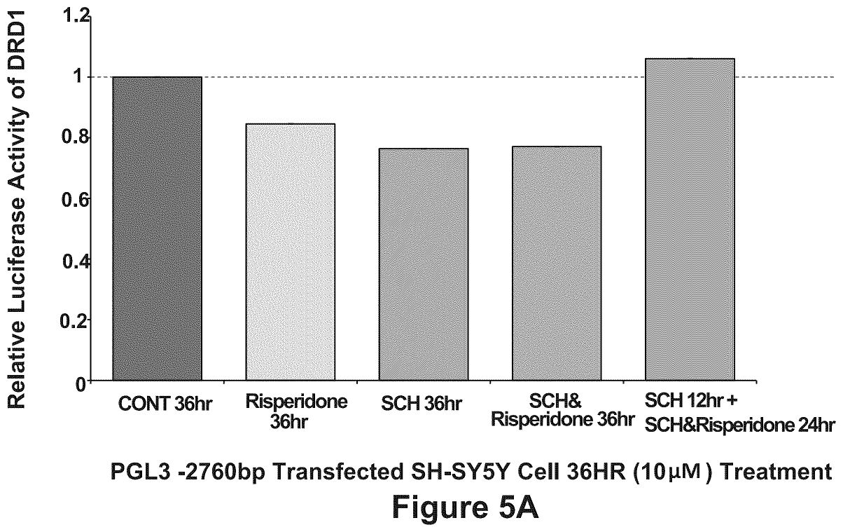

Referring to FIGS. 3A-3E of the drawings, the results show that Haloperidol attenuated SKF81297-increased DRD1 mRNA and protein expression in SH-SY5Y cells.

SKF81297 treatment for 18 and 24 hrs increased human DRD1 mRNA expression (FIG. 3A). Whereas Haloperidol did not alter DRD1 mRNA expression after either 18 or 24 hrs. Haloperidol co-treatment for 12 hrs completely diminished SKF81297-increased DRD1 mRNA expression (FIG. 3A). SKF81297 treatment for 36 hrs increased membrane DRD1 protein levels more than 2-fold (FIG. 3B and 3C). Haloperidol co-treatment for 24 hrs markedly attenuated SKF81297-increased DRD1 membrane protein level. Immunocytostaining showed that SKF81297 increased human DRD1-positive cell counting almost 5-fold as compared to control (FIG. 3D). Such increase is completely abolished following Haloperidol cotreatment.

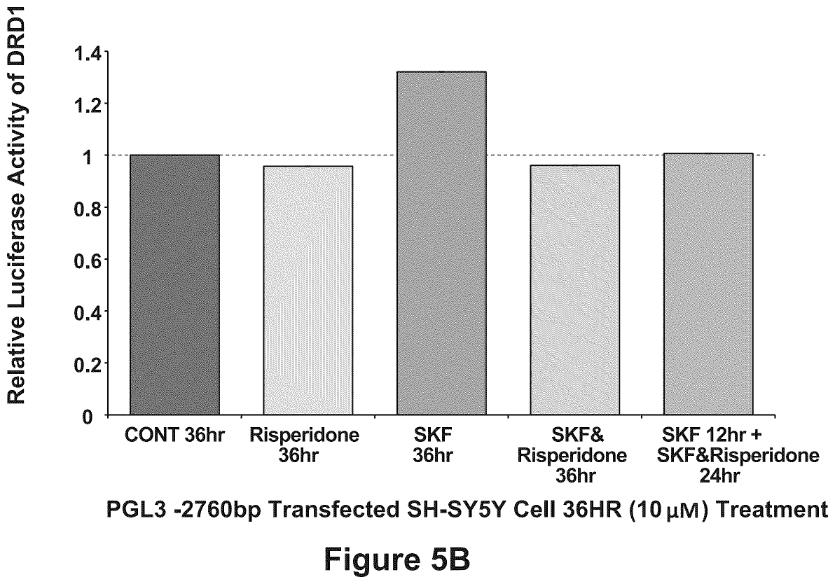

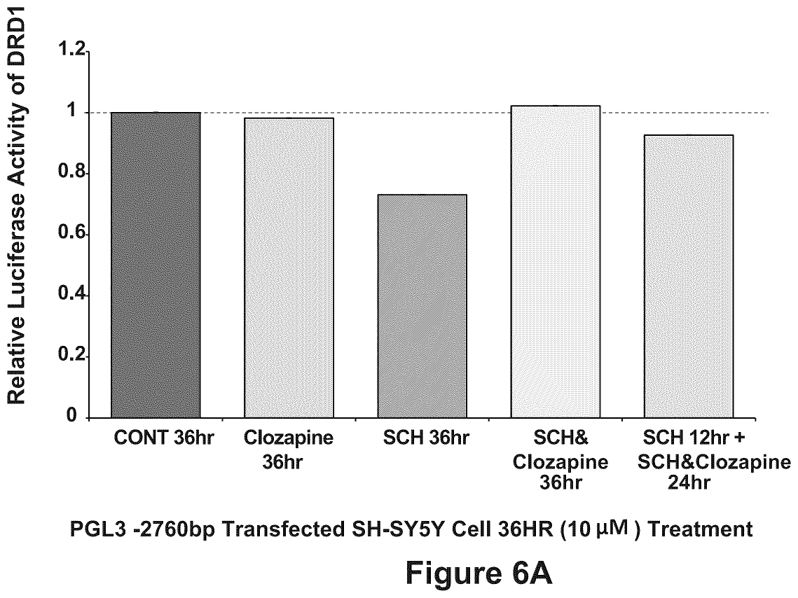

Referring to FIGS. 4A-4B of the drawings, the results show that Haloperidol reversed SCH23390/SKF21897-altered DRD1 promoter activity.

Engineered recombinant pGL3 reporter constructs containing 2.8-kb human DRD1 gene promoter were transfected into SH-SY5Y cells. Luciferase reporter assays showed that Haloperidol did not change human DRD1 gene promoter activity (FIG. 4A). In contrast, SCH23390 treatment for 36hrs trans-repressed DRD1 promoter activity, which is reversed by haloperidol cotreatment for 24 or 36 hrs (FIG. 4A). In addition, SKF81297 treatment for 36hrs trans-activated DRD1 promoter activity (FIG. 4B), which is markedly attenuated by haloperidol co-treatment for both 24 and 36 hrs.

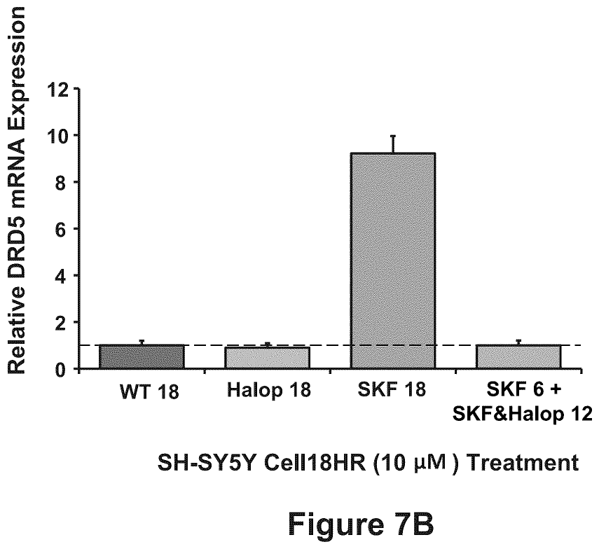

Similarly, DRD5 mRNA regulation by SCH+Haloperidol and SKF+Haloperidol are also studied. The results are shown in FIG. 7A and 7B of the drawings. The results show that the effect of SCH+Haloperidol and SKF+Haloperidol on DRD5 are similar to that of the effect of DRD1.

The effects of several antipsychotics on DRD1 promoter activity, including Risperidone, Clozapine and Cariprazine are also studied.

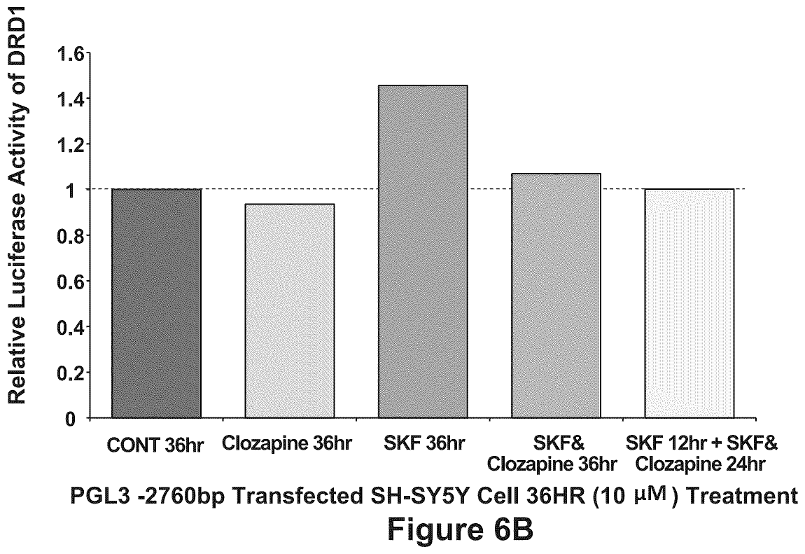

Referring to FIGS. 5A and 5B of the drawings, the results show that Risperidone trans-stabilize SCH23390/SKF81297-altered DRD1 promoter activity.

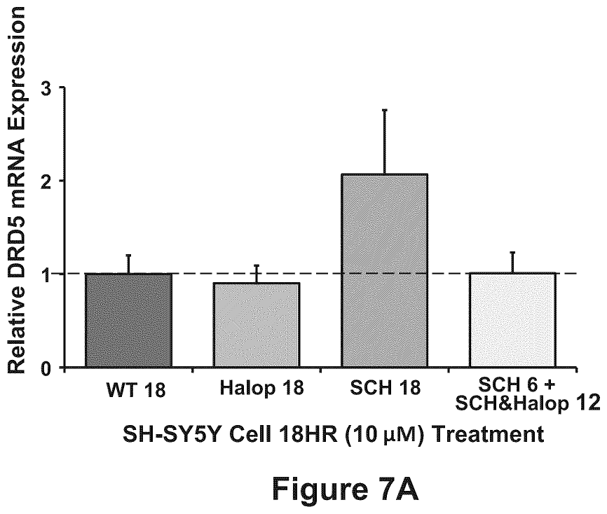

Referring to FIGS. 6A and 6B of the drawings, the results show that Clozapine trans-stabilize SCH23390/SKF81297-altered DRD1 promoter activity.

The results also show that Cariprazine trans-stabilize SCH23390/SKF81297-altered DRD1 promoter activity.

In summary, our results show that:
1) 10 μM Haloperidol does not alter DRD1 expression. In contrast, 10 μM SCH23390 decreases, whereas 10 μM SKF81297 increases DRD1 mRNA and protein expression in a time-dependent manner.
2) SCH23390-decreased DRD1 mRNA and protein expression is reversed by Haloperidol co-treatment.
3) Up-regulated DRD1 mRNA and protein expression by SKF-81297 is attenuated by Haloperidol co-treatment.
4) Dual luciferase reporter assays demonstrates that SCH23390 transactivates whereas SKF81297 represses activity of 2.8-kb human DRD1 gene promoter. In addition, Haloperidol reversed either SCH23390 or SKF81297-altered human DRD1 promoter activity.
5) Similar to Haloperidol, Risperidone and Clozapine reverse and normalize either SCH23390 or SKF81297-altered human DRD1 promoter activity. These data indicates that the antipsychotics can bidirectionally normalize chemically altered human DRD1 expression, by targeting its upstream promoter sequence. These findings support why most antipsychotics are effective in treating bipolar disorders and substance-induced psychotic disorder, in which DRD1 expression is genetically normal but is chemically altered.

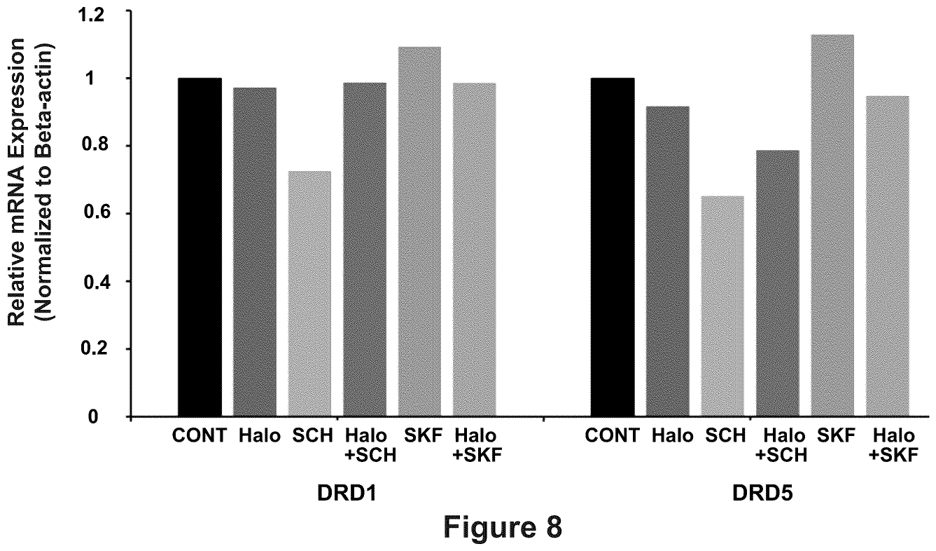

Referring to FIG. 8 of the drawings, the relative mRNA expression of DRD1 and DRD5 in SHSY5Y cells are illustrated. The SHSY5Y cells are cultured in T-25 flask (1×10⁶ cells/T-25 flask) and treated with the followings:
(a) Halo: Haloperidol (10 μM) for 20 hrs; (b) SCH: SCH23390 (10 μM) for 20 hrs; (c) SKF: SKF21987 (10 μM) for 20 hrs; (d) Halo+SCH: SCH 23390 (10 μM) and Halo (10 μM), of which SCH is pretreated for 6 hrs, and then SCH 23390 and Halo are cotreated for another 14 hrs; (e) Halo+SKF: SKF 81297 (10 μM) and Halo (10 μM), of which SKF is pretreated for 6 hrs, and then SKF 81297 and Halo are cotreated for another 14 hrs; and (f) CONT: DMSO only as a Control treatment.

After treatment, the SHSY5Y cells are harvested and processed for RT-PCR analysis. The data are expressed as the mean of three individual samples per treatment.

Clinical study of the DRD1 and DRD5 genomic sequence variation in human subjects

Genomic sequence of human DRD1 and DRD5 are analyzed by using Illumina MiniSeq instrument. The genomic DNA is extracted from human blood using Qiagen Blood & Cell Culture DNA Kits according to the manufacturer's instructions (Qiagen, Beverly, MA). The genomic DNA sequence data are analyzed by using Illumina apps DNA Amplicon and DRAGEN Amplicon.

Three exemplary human subjects are selected to demonstrated the relationship between the variation of human DRD1 genomic DNA and the mental disorders.

The results are summarized as follows in Table 1: Variation of human DRD1 genomic DNA (including 7-kb 5′-regulatory sequence, gene coding sequence, and 1-kb 3′-regulatory sequence) in the three human subjects with mental disorders

| | DRD1 | | | |
|---|---|---|---|---|
| | Chr. 5 (GRCh37/hg19) | | | |
| | | Variation Frequency | | |
| | | Subject | Subject | Subject |
| Location | rs No. | No. 1 | No. 2 | No. 3 |
| 174866920 | rs251937 | 1 | 1 | 0.998 |
| 174867169 | rs12518222 | 1 | | |
| 174867205 | rs144667196 | | | |
| 174867212 | rs139841523 | | | |
| 174867899 | rs4867798 | | | |
| 174868700 | rs686 | 0.995 | 0.999 | 0.997 |
| 174868840 | rs155417 | 0.999 | 0.999 | 1 |
| 174869905 | synonymous_variant | | | |
| 174870150 | rs4532 | 0.999 | 0.999 | 0.998 |
| 174870196 | rs5326 | 0.999 | | |
| 174870902 | rs265981 | 0.995 | 0.995 | 0.987 |
| 174871354 | rs35916350 | 0.514 | | |
| 174872132 | rs10078714 | 0.463 | | |
| 174872204 | rs10063995 | 0.489 | | |
| 174872320 | rs10078866 | 0.499 | | |
| 174872883 | rs267410 | 0.543 | 0.998 | 0.997 |
| 174873174 | rs35469033; rs397882632 | | 0.363 | 0.373 |
| 174873658 | rs10052729 | 0.487 | | |
| 174874623 | rs147998478; rs11268110 | 0.214 | | |
| 174874844 | rs6871442 | 0.487 | | |
| 174875166 | rs74473267 | 0.504 | | |
| 174875472 | rs267412 | 0.545 | 1 | 1 |
| 174875608 | rs267413 | 0.506 | 0.997 | 0.998 |
| 174875978 | rs72815442 | | | |
| 174876002 | rs2168631 | 0.469 | | |
| 174876055 | rs35968413 | 0.458 | | |
| 174876138 | rs4008100; rs397822366; | 0.761 | 0.772 | 0.761 |
| | rs748989617 | | | |
| 174876401 | rs6878159 | 0.475 | | |
| 174876427 | rs6882300 | 0.477 | | |
| 174876845 | rs112471953; rs539212301 | | | |
| 174877271 | rs267415 | 1 | 0.999 | 1 |
| 174877400 | rs74445963 | | 0.506 | 0.509 |
| 174877540 | rs10076244 | 0.502 | | |
| 174877945 | rs74580650 | | | |

In our clinical studies, at least one variation in DRD1 or DRD5 is found in human subject with mental disorder. The results also show that when the number of variation increases, the level of seriousness of the mental disorder increases.

In other words, the mental disorder can be identified quantitatively by locating at least one variation in DRD1 or at least one variation in DRD5. More serious type of mental disorder can be identified by two or more variations in DRD1 and DRD5.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of treatment of mental disorder of a subject, comprising the steps of: normalizing a DRD1 level and a DRD1 expression of the subject to a preset standard level.

2. The method of treatment of mental disorder of a subject according to claim 1, further comprising the steps of: increasing or decreasing the DRD1 constitutive expression of the subject by targeting an upstream promoter sequence of DRD1.

3. The method of treatment of mental disorder of a subject according to claim 1, further comprising the steps of: normalizing a DRD5 level and a DRD5 expression of the subject to a preset standard level.

4. The method of treatment of mental disorder of a subject according to claim 3, further comprising the steps of: increasing or decreasing the DRD1 or DRD5 constitutive expression of the subject by targeting an upstream promoter sequence of DRD1 or DRD5; and attenuating a promoter activity of DRD1 or DRD5 by using an antipsychotic which directly targets the DRD1 or DRD5 promoter.

5. The method of treatment of mental disorder of a subject according to claim 3, wherein the antipsychotic is selected from the group consisting of Haloperidol, Risperidone, Clozapine and Cariprazine.

6. The method of treatment of mental disorder of a subject according to claim 4, wherein the antipsychotic is selected from the group consisting of Haloperidol, Risperidone, Clozapine and Cariprazine.

7. The method of treatment of mental disorder of a subject according to claim 1, wherein the subject has an impaired constitutive expression of DRD1 gene.

8. The method of treatment of mental disorder of a subject according to claim 2, wherein the subject has an impaired constitutive expression of DRD1 gene.

9. The method of treatment of mental disorder of a subject according to claim 4, wherein the subject has an impaired constitutive expression of DRD1 or DRD5 gene.

10. The method of treatment of mental disorder of a subject according to claim 2, wherein the mental disorder comprises schizophrenia, autism, depression, mania and bipolar disorders.

11. The method of treatment of mental disorder of a subject according to claim 4, wherein the mental disorder comprises schizophrenia, autism, depression, mania and bipolar disorders.

12. A method of treatment of mental disorder of a subject, comprising the steps of: bidirectionally normalizing an overall expression level of DRD1, DRD5, or DRD1 and DRD5 of the subject to a preset standard level.

13. The method of treatment of mental disorder of a subject according to claim 12, further comprising the steps of: attenuating a promoter activity of DRD1/DRD5 by using an antipsychotic which directly targets the DRD1 and/or DRD5 promoter.

14. The method of treatment of mental disorder of a subject according to claim 13, wherein the antipsychotic is selected from the group consisting of Haloperidol, Risperidone, Clozapine and Cariprazine.

15. The method of treatment of mental disorder of a subject according to claim 13, wherein the mental disorder comprises schizophrenia, autism, depression, mania and bipolar disorders.

16. The method of treatment of mental disorder of a subject according to claim 14, wherein the mental disorder comprises schizophrenia, autism, depression, mania and bipolar disorders.

17. The method of treatment of mental disorder of a subject according to claim 13, further comprising the steps of: providing a sequence database in which polymorphism of DRD1 and/or DRD5 promoter sequences are related to medication efficacy; and personizing medication screening of antipsychotic drugs regarding efficacy based on the sequence database.

18. A quantitative screening method of mental disorder of a subject, comprising the steps of:
carrying out genomic sequencing of DRD1 and DRD5 genes (including 7-kb 5′-upstream sequence, protein coding sequence, and 2-kb 3′-downstream sequence) of a plurality of subjects to establish a relationship between a mental disorder and a variant distribution database (including a type, a location and a frequency of each gene variant) in the DRD1 and DRD5 genes;
sorting out one or more variant that is highly related to one mental disorder to develop a target gene sequence of DRD1 and DRD5 genes, which can be used to determine a risk of developing the mental disorder;
performing the target gene sequence of DRD1 and DRD5 genes in a target subject to determine the risk of developing the mental disorder in the target subject.

19. The quantitative screening method of mental disorder of a subject, wherein more than two variants are related to one mental disorder.

20. The quantitative screening method of mental disorder of a subject according to claim 19, wherein polymorphism of DRD1 and/or DRD5 promoter sequences which are related to clinically diagnosed mental disorders are included in the variant distribution database.

21. The quantitative screening method of mental disorder of a subject according to claim 19, wherein the method is a prenatal screening method and polymorphism of DRD1 and/or DRD5 promoter sequences which are related to the risk of clinically diagnosed mental disorders are included in the variant distribution database.

22. A quantitative screening test of human DRD1 and DRD5 expression in a cell of a human subject, comprising the steps of:
establishing normal ranges of human DRD1 and DRD5 expression in the cell;
determining the human DRD1 and DRD5 expression in the cell of the human subject; and
comparing the human DRD1 and DRD5 expression in the cell of the human subject with the normal ranges to determine a mental status of the human subject,
wherein the cell is selected from the group consisting of mucosal cell, skin cell, and blood cells.

23. A quantitative method of screening new medications of mental disorder, comprising the steps of:
bidirectionally normalizing an overall expression level of DRD1 and/or DRD5 of the subject to a preset standard level,
wherein the mental disorder comprises schizophrenia, autism, depression, mania and bipolar disorders,
wherein the sample sequence comprises at least one variations when compared to the standard sequence database.


SEQUENCE LISTING (4 sequences)

SEQ ID NO: 1 - DNA, 20 bp, Homo sapiens
  source 1..20: mol_type = genomic DNA
ccatcacacaaaacggtcag

SEQ ID NO: 2 - DNA, 20 bp, Homo sapiens
  source 1..20: mol_type = genomic DNA
gtgtgttggaaagcagcaga

SEQ ID NO: 3 - DNA, 20 bp, Homo sapiens
  source 1..20: mol_type = genomic DNA
gcaattattccccatgaacg

SEQ ID NO: 4 - DNA, 19 bp, Homo sapiens
  source 1..19: mol_type = genomic DNA
gcctcactaaaccatccaa